(19) United States
(12) Patent Application Publication
(10) Pub. No.: US 2008/0262481 A1
(43) Pub. Date: Oct. 23, 2008

(54) Hand Skin Care Apparatus

(76) Inventor: Hyun Jin Cho, Seoul (KR)

Correspondence Address: OSTROLENK FABER GERB & SOFFEN, 1180 AVENUE OF THE AMERICAS, NEW YORK, NY, 100368403, US

(21) Appl. No.: 11/817,154

(22) PCT Filed: Mar. 18, 2005
(86) PCT No.: PCT/KR05/00802

§ 371 Date: Aug. 27, 2007

(30) Foreign Application Priority Data
Mar. 10, 2005 (KR) 10-2005-0020293

(51) Int. Cl. A61B 18/04

(52) U.S. Cl. 606/9

(57) ABSTRACT

The present invention relates to a hand skin care apparatus for performing multiple functions of drying and cosmetically treating hands, caring hand skin, aiding health, therapeutic activation, and fomentation, therapeutic activation, and fomentation, and employs a cover pivotally opened and closed about a hinge shaft of a main body for sucking air, projecting infrared rays, heating using infrared rays, and projecting laser beams. The introduced air passes through the infrared ray projector and is blown to where a user's hands are placed such that hands are rapidly dried by hot air and heat, infrared rays, and infrared rays generated by the infrared ray projector and the infrared exothermic device prevent and remove the inhabitance of bacteria causing various diseases on the hands. Thus, a user can maintain his/her hands at sanitary state. Due to the increased temperature, the apparatus of the present invention exhibits fomentation and the far infrared rays are helpful to hand skin care and health. Moreover, the low-level laser beam is projected to hands to activate the treatment of affected parts and various hand diseases, resulting in aiding health. Since a single apparatus performs various and multiple functions, the hand skin care apparatus is variously used, has various effects. Moreover, economic value and utility of the hand skin care apparatus are enhanced.

### TECHNICAL FIELD

The present invention relates to a hand skin care apparatus for performing multiple functions of drying hands, cosmetically treating hands, caring hand skin, aiding health, therapeutic activation, and fomentation.

### BACKGROUND ART

Recently, numerous food poisoning incidents due to unsanitary treatment of food occur throughout the world on a daily basis. Particularly, since more than 13% of food poisoning incidents occur due to contamination by microorganisms present on the hands, good hygiene of persons charged with treating foods is essential.

Some apparatuses for keeping hands sanitary are known, and, among these apparatuses, air dryers serve to jet high-pressure air to dry hands washed with water by removing moisture from the wet hands. However, since the air dryer has only a function of drying hands using air jet, the use of the air dryer is limited, and since the air jet is directly discharged out, the air jet is not concentrated to the wet hands and power consumption is increased.

Alternative automatic hand washing apparatuses are available. Automatic hand washing apparatuses are used without soup and/or towels, are capable of being used as a mobile washstand in locations where there is insufficient space to install the washstand or there is no washstand. However, since the automatic hand washing apparatus has only a function for washing and drying hands, the automatic hand washing apparatus has limited range of applications. Moreover, since the automatic hand washing apparatus is very large, space for installing the automatic hand washing apparatus must be guaranteed when the automatic hand washing apparatus is connected to the water supply. Since a hand dryer employs the structure of the conventional air dryer, hot wind is not concentrated to wet hands, heat loss is increased and a high capacity heater is required. Thus, power consumption is increased.

Infrared rays remove bacteria causing various diseases and expand the capillaries, thereby improving blood circulation and cellular tissues,

accelerating metabolism, preventing fatigue, promoting diaphoresis, alleviating pains, removing heavy metals, promoting deep sleep, eliminating odors, exhibiting bactericidal effects, dehumidifying, and purifying air, and various apparatuses using infrared rays are known. However, the apparatuses using infrared rays is limited to aid treatment, and there is no apparatus for intensively performing functions on hands and having multiple functions.

Moreover, there are various apparatuses using infrared rays such as beds, foot massagers, dishcloth dryers, thermotherapy devices, and sitz bathes. However, each of these apparatuses performs drying or heating simply using heat or simple functions using effects of far infrared ray.

In addition, low-level lasers are effective for skin care such as treatment of acne, hand eczema, pityriasis, or the like, wrinkle removal, and bacteria removal, and apparatuses using lasers, such as an acne therapy system, a skin care device, and a laser peeler are known. However, these apparatuses have simple functions and there is no apparatus for intensively performing functions on hands and having multiple functions.

### DISCLOSURE OF INVENTION

### Technical Problem

Therefore, the present invention has been made in view of the above and/or other problems, and it is an object of the present invention to provide a hand skin care apparatus for performing multiple functions such as drying moisture of hands, cosmetic treat of hands, hand skin care, health aide, therapeutic activation, and fomentation.

### Technical Solution

In accordance with the present invention, the above and other objects can be accomplished by a provision of a hand skin care apparatus including: a suction part (12) for sucking ambient air into a main body (10) by a suction fan (12A) and a grill (12B) fixed to a rear inner side of the main body (10); an infrared ray projector (13) including an infrared ray generator (13C) fixed to an inner central portion of a pipe-shaped insulator (13A) by the pipe-shaped insulator (13A) and a support (13B) to emit heat and infrared rays, and a protective net (13D) installed to the front side of the infrared ray generator (13C); an infrared exothermic device (14) installed directly below a cover (20) to be connected to the infrared ray projector (13) to emit heat and far infrared rays; a control panel (16) exposed over the surface of a front side (15) of the main body (10) connected to the infrared exothermic device (14) to control operation of the hand skin care apparatus; a laser generator (17) fixed in the front side (15) and projecting laser beams on hands; the cover (20) pivotally coupled to the main body (10) by a hinge shaft (H) to be opened and closed; a hand accommodating part (30) formed in the cover (20); a hand insertion opening (40) for guiding a user to insert his/her hands into the hand accommodating part (30); a pair of detecting sensors (50 and 50′) fixed to the inner circumference of the hand insertion opening (40) to detect the insertion of the hands to control the hand skin care apparatus; and a locking structure (60) installed to the front side (15) and the cover (20) to selectively lock the cover (20); wherein the infrared rays, the far infrared rays, and the laser beam are projected to the user's hands inserted into the hand accommodating part (30).

Preferably, the infrared ray generator (13C) includes an infrared ray lamp (13C1) for emitting heat and far infrared rays.

The infrared ray generator (13C) includes a halogen lamp (13C3) for emitting heat and far infrared rays.

The infrared ray generator (13C) is made by winding a heating wire (13C8) around an outer circumference of a quartz glass tube (13C4) accommodating kaoline, yellow soil, or the combination of the kaoline and the yellow soil.

The infrared ray generator (13C) is made by winding a heating wire (13C10) around an outer circumference of a composite ceramic body (13C9) manufactured in the form of a cylinder, a rectangular rod, or a hollow cylinder.

The main body (10) is formed with a plurality of insertion holes (13C2) formed in regular intervals to insert and install infrared lamps (13C1).

The halogen lamp (13C3) is disposed in a pipe-shaped composite ceramic body (13C6).

The infrared exothermic device (14) includes upper and lower insulating plates (14B and 14C), a heating plate (14A) disposed between the upper and lower insulating plates (14B and 14C), a surface member (14D) disposed on the upper insulating plate (14B) to emit infrared rays, and an adiabatic plate (14E) disposed on the lower insulating plate (14C) to block heat transfer.

The infrared exothermic device (14) includes upper and lower insulating plates (14B and 14C), a plane heater (14F) disposed between the upper and lower insulating plates (14B and 14C), a surface member (14D) disposed on the upper insulating plate (14B) to emit infrared rays, and an adiabatic plate (14E) disposed on the lower insulating plate (14C) to block heat transfer.

The infrared exothermic device (14) includes a halogen lamp (14G) attached to the lower surface of a surface member (14D), and a reflecting plate (14H) disposed under the halogen lamp (14G), and an adiabatic member (14I) disposed under the reflecting plate (14H).

The cover (20) includes a ceramic wall (22) disposed at the inner wall of the cover (20) and having a plurality of grooves (23) formed in the longitudinal direction.

The cover (20) includes a transparent window (24) formed at the upper side of the cover (20) in a predetermined size to observe the hands inserted into the hand accommodating part (30).

The locking structure (60) of the cover (20) includes a locking device (63) including a handle (61) protruded outside and an insertion guiding slope (62), a spring (64) for returning the locking device (63) to its original position, and a locking groove (65) formed at the front side (15) and into which the locking device (63) is selectively inserted.

The hand insertion opening (40) has a pair of hand insertion openings (40) symmetrically formed at front right and left sides of the hand accommodating part (30).

The hand insertion opening (40) has a single hand insertion opening (40) formed at a central portion of the front side (15) to be communicated to the inside of the hand accommodating part (30).

The hand skin care apparatus further includes a support (70) coupled to the lower side of the main body (10) by a hinge shaft (71) and a torsion spring (72) to be elastically pivoted.

The hinge shaft (H) of the main body (10) includes a hinge shaft gear (66B) and a torsion spring (66A) installed thereto, and the hinge shaft gear (66B) is engaged with a reduction gear (66C) fixed to the cover (20) or the rear side of the main body (10).

### Advantageous Effects

As described above, the hand skin care apparatus of the present invention employs the cover to be pivotally opened and closed about the hinge shaft installed to the main body having the functions of sucking air therein, projecting infrared rays, heating using the infrared rays, projecting laser beam. The air introduced from outside by the suction fan passes through the infrared ray projector and is blown to where user's hands are placed such that wet hands are rapidly dried due to the hot air and heat, infrared rays, and infrared rays generated by the infrared ray projector and the infrared exothermic device prevent and remove the inhabitance of bacteria causing various diseases on the hands. Thus, a user can maintain his/her hands at sanitary state. Due to the increased temperature, the hand skin care apparatus of the present invention exhibits fomentation and the far infrared rays are helpful to hand skin care and health. Moreover, the low-level laser beam is projected to hands to activate the treatment of affected parts and various hand diseases, resulting in aiding health. Since a single hand skin care apparatus performs various and multiple functions, the hand skin care apparatus is variously used, and has various effects. Moreover, economic value and utility of the hand skin care apparatus are enhanced.

### BRIEF DESCRIPTION OF THE DRAWINGS

The object and advantages of the present invention will become apparent and more readily appreciated from the following description of preferred embodiments, taken in conjunction with the accompanying drawings, in which:

FIG. 1 is a perspective view illustrating a hand skin care apparatus according to the preferred embodiment of the present invention;

FIG. 2 is a sectional view illustrating an internal structure and operation of the hand skin care apparatus in FIG. 1;

FIG. 3 is a side view illustrating the opening and closing operation of a cover employed in the hand skin care apparatus according to the preferred embodiment of the present invention;

FIG. 4 is a vertical sectional view of the cover employed in the hand skin care apparatus according to the preferred embodiment of the present invention;

FIG. 5 is a front view illustrating a control panel employed in the hand skin care apparatus according to the preferred embodiment of the present invention;

FIG. 6 is a front view illustrating a hand skin care apparatus, having a single hand insertion opening formed at the front side thereof, according to another preferred embodiment of the present invention;

FIG. 7 is a side view illustrating a hand skin care apparatus, in which a main body is tilted by a support, according to still another preferred embodiment of the present invention;

FIG. 8 is a side view illustrating a hand skin care apparatus, in which a main body is tilted by a support, according to still another preferred embodiment of the present invention;

FIG. 9 is a sectional view illustrating an infrared ray lamp as an infrared ray projector employed in the hand skin care apparatuses according to the preferred embodiments of the present invention;

FIG. 10 is a sectional view of the infrared ray lamp taken in a direction different from the direction of the sectional view in FIG. 9;

FIG. 11 is a sectional view illustrating the infrared ray lamp applied to the hand skin care apparatuses according to the preferred embodiments of the present invention;

FIG. 12 is a sectional view illustrating a halogen lamp as the infrared ray projector in the hand skin care apparatuses according to the preferred embodiments of the present invention;

FIG. 13 is a sectional view illustrating a combination of a halogen lamp and composite ceramic as an infrared ray projector employed in the hand skin care apparatuses according to the preferred embodiments of the present invention;

FIG. 14 is a sectional view illustrating another example of the infrared ray projector employed in the hand skin care apparatuses according to the preferred embodiments of the present invention;

FIG. 15 is a sectional view illustrating an electric heating coil wound around a composite ceramic as an infrared ray projector employed in the hand skin care apparatuses according to the preferred embodiments of the present invention; and

FIGS. 16 to 18 are views illustrating examples of an infrared ray exothermic part employed in the hand skin care apparatuses according to the preferred embodiments of the present invention.

### BEST MODE FOR CARRYING OUT THE INVENTION

Hereinafter, the preferred embodiments of the present invention will be described in detail with reference to the accompanying drawings.

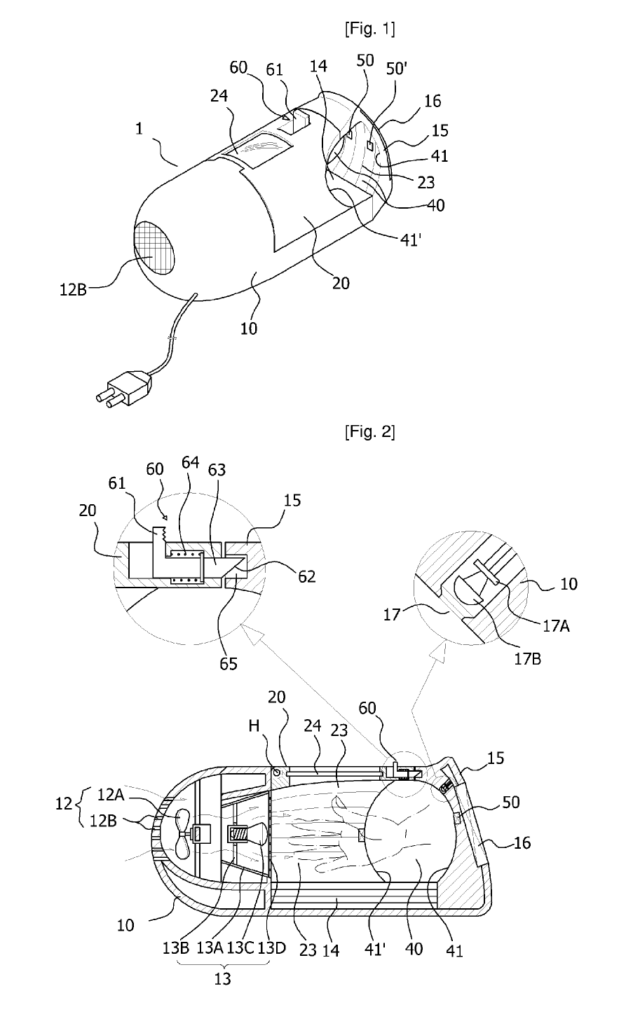

FIG. 1 is a perspective view illustrating a hand skin care apparatus according to the preferred embodiment of the present invention, and FIG. 2 is a sectional view illustrating the internal structure and operation of the hand skin care apparatus in FIG. 1.

The hand skin care apparatus according to the preferred embodiment of the present invention includes a main body 10, a cover 20 connected to the main body 10 by a hinge shaft H to be opened and closed, and a hand accommodating part 30 formed in the cover 20. Hereinafter, the hand skin care apparatus will be described by defining a side of the main body 10 near to a user as a front side 15 and the other side as a rear side of the main body 10.

The main body 10 includes a suction part 12 for sucking ambient air into the main body 10 through a suction fan 12A and a grill 12B, which are fixed to the rear side of the main body 10, an infrared ray projector 13 having an infrared ray generator 13C fixed at a central portion of a pipe-shaped insulator 13A by the insulator 13A and a support 13B, which are fixed at the front side of the suction part 12, to emit heat and infrared rays, and a protecting net 13D installed in front of the infrared ray generator 13C to protect hands inserted into the hand accommodating part 30 from being burned by heat generated from the infrared ray generator 13C, an infrared exothermic device 14 installed directly below the cover 20 to be connected to the infrared ray projector 13 and to emit heat and far infrared rays, a control panel 16 installed to be exposed from the surface of the front side 15 of the main body 10 connected to the infrared exothermic device 14 to control overall operation of the hand skin care apparatus, and a laser generator 17 fixed in the front side 15 of the main body 10 to project a laser to the hands. Optionally, a plurality of laser generators 17 may be installed any places including the rear inside of the cover 20.

FIGS. 9 to 15 show various examples of the infrared ray generator 13C employed in the hand skin care apparatus according to the preferred embodiments of the present invention.

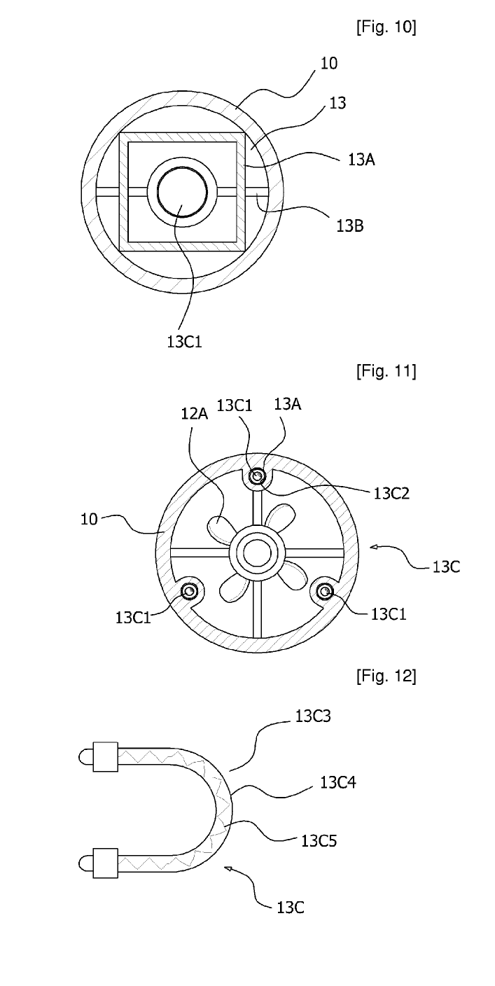

Firstly, hand skin care apparatuses in FIGS. 9 to 11 employ an infrared lamp 13C1. The infrared ray lamp 13C1 emits thermal energy and infrared rays when the infrared ray lamp 13C1 is turned on. The infrared rays are far infrared rays with a wavelength of 5.6□ to 1,000□ helpful to health. The infrared rays are projected without adjusting voltage level depending on a user's wish such that heat is absorbed into human body, or are projected by properly adjusting voltage level such that the infrared rays permeated deep into human body without skin burn. By doing so, the infrared rays are also helpful for warming and therapy, to remove bacteria, and to accelerate blood circulation and produce cellular tissues due to capillary vessels expanded by the projected infrared rays. A single infrared ray lamp 13C1 is installed at the central portion of the insulator 13A as shown in FIG. 10, or, as shown in FIG. 11, a plurality of infrared ray lamps 13C1 may be installed to the main body 10 having a plurality of insertion holes 13C2 formed at the inner wall of the main body 10 at regular intervals.

Secondarily, as shown in FIG. 12, a halogen lamp 13C3 is employed in the hand skin care apparatus according to the preferred embodiments of the present invention. The halogen lamp 13C3 is structured such that a tungsten filament 13C5 is inserted into a quartz glass tube 13C4 formed in a U-shape, an I-shape, a ring-shape, or a spiral-shape, inert gas such as nitrogen or argon and a small amount of a halogen are injected into the quartz glass tube 13C4, and the quartz glass tube 13C4 is sealed. The halogen lamp 13C3 has excellent durability, high heating value, and low power consumption. The tungsten filament cannot be replaced with a carbon filament or graphite filament. If the surface of the quartz glass tube 13C4 is coated with a material capable of emitting far infrared rays, the infrared rays and the far infrared rays are emitted from the surface of the quartz glass tube 13C4.

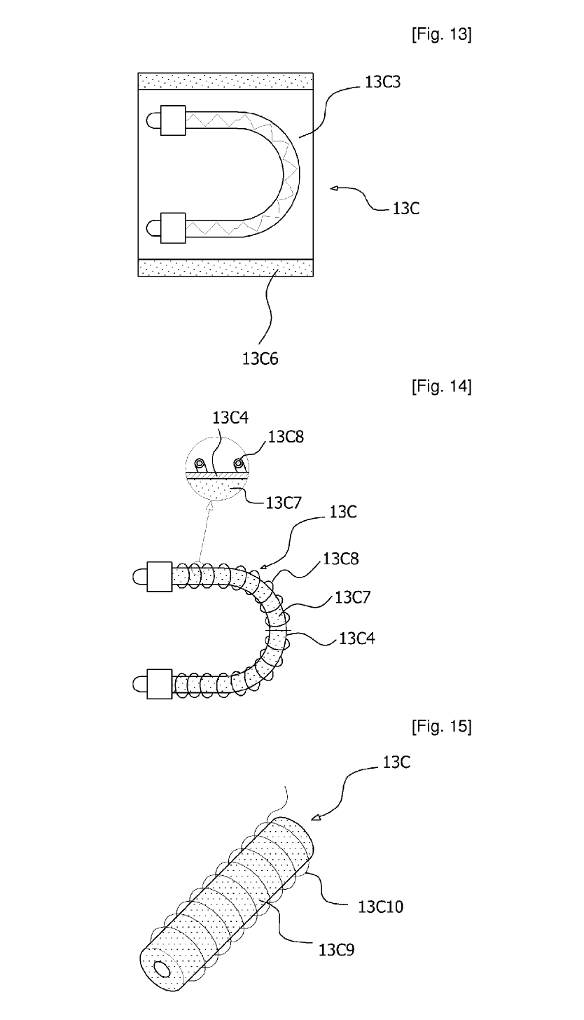

Thirdly, the hand skin care apparatus in FIG. 13 employs a pipe-shaped composite ceramic body 13C6 surrounding the halogen lamp 13C3 constructed as shown in FIG. 12 to emit the far infrared rays from the composite ceramic 13C6 due to exothermic reaction of the halogen lamp 13C3.

The composite ceramic body 13C6 is made of a composite ceramic or a composite sintered ceramic. The composite ceramic is made by mixing a ceramic material such as mullite, ferrite, or aluminum titanate, with mineral substances such as sericite, enterolith, pyrophylite, bentonite, serpentine, quartz porphyry, quartz, monzonite, gneiss, rhyolite, or tuff, and the composite sintered ceramic is made by mixing several ceramic materials with the mineral substances. Since the composite ceramic and the composite sintered ceramic have undergone plastic deformation at temperatures higher than 1,250 degrees centigrade, the composite ceramic and the composite sintered ceramic are light and emit the far infrared rays highly effectively.

Fourthly, in the hand skin care apparatus as shown in FIG. 14, an heating wire 13C8 is wound around the outer surface of the quartz glass tube 13C in which kaoline, yellow soil, or a mixture 13C7 of kaoline and yellow soil are accommodated, such that the kaoline or the yellow soil in the quartz glass tube 13C receives heat from the heating wire 13C8 and emits infrared rays.

Fifthly, in the hand skin care apparatus in FIG. 15, a composite ceramic body 13C9 is made of the same material as the composite ceramic body 13C6 in FIG. 13 and is manufactured in the form of a cylinder, a rectangular rod, or a hollow cylinder. A heating wire 13C10 such as a steel chrome wire, nichrome wire, or the like is wound around the outer circumference of the composite ceramic body 13C9 such that the composite ceramic body 13C9 emits the far infrared rays using heat generated by the heating wire 13C10.

Meanwhile, the hand skin care apparatus of the present invention employs three examples of the infrared exothermic device 14.

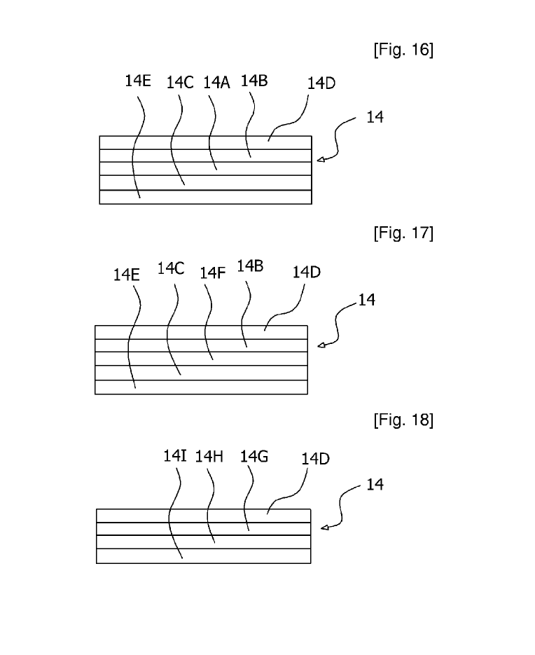

Firstly, as shown in FIG. 16, the infrared exothermic device 14 includes a heating plate 14A disposed at the intermediate portion thereof, upper and lower insulating plates 14B and 14C disposed on and under the heating plate 14A, a surface member 14D disposed on the upper insulating plate 14B to emit the far infrared rays, and an adiabatic plate 14E disposed under the lower insulating plate 14C to block heat transfer, and adopts a heating system for heating using a heating plate. The heating plate 14A may be a heating coil, an electric panel, an electric pipe, a coil heater, or the like. When electricity is applied to the heating plate 14A, heat is transferred to the surface member 14D to emit the far infrared rays to hands inserted into the hand skin care apparatus according to the preferred embodiment of the present invention.

Secondarily, instead of the heating plate 14A depicted in FIG. 16, as shown in FIG. 17, the infrared exothermic device 14 adopts a heating system for heating and emitting using a plate heater 14F. The plane heater 14F may be a plate heater such as a metal thin film etching plane heater, a carbon black thick film plane heater, a carbon black polymer plane heater, or a carbon fiber plane heater. When electricity is applied to the plane heater 14F, heat is transferred to the surface member 14D to emit the infrared rays to hands in the hand skin care apparatus according to the preferred embodiments of the present invention.

Thirdly, an infrared exothermic device 14 in FIG. 18 includes a halogen lamp 14G, which is attached to the lower side of the surface member 14D, and a reflecting plate 14H and an adiabatic member 14I disposed under the halogen lamp 14G in turn. When electricity is applied to the halogen lamp 14G, heat is transferred to the surface member 14D such that infrared rays generated from the surface member 14D are projected to hands in the hand skin care apparatus according to the preferred embodiments of the present invention. The halogen lamp 14G, like the halogen lamp in FIG. 12, may be structured by a tungsten filament inserted into a quartz glass tube formed in a U-shape, an I-shape, a ring-shape, or a spiral-shape, by injecting inert gas such as nitrogen or argon and a small amount of halogen material injected into the quartz glass tube 13C4, and sealing the quartz glass tube.

The surface member 14D may be made of various materials, such as a plate made of raw rock such as jade, quartz porphyry, sericite, enterolith, pyrophylite, bentonite, serpentine, alunite, quartz, monzonite, gneiss, rhyolite, tuff, germanium, illite, amethyst, tourmaline, zeolite, white jade, olivine, or biostone, a metal plate, a ceramic tempered glass, the composite ceramic body, a ceramic coating film processed plate, a yellow plate subjected to high temperature plastic deformation.

FIG. 5 is a front view illustrating a control panel 16 employed in the hand skin care apparatus according to the preferred embodiment of the present invention. The control panel 15 basically includes a power switch 16A, and may further include a blowing time adjusting switch 16B for driving the suction fan 12A to blow air, the infrared ray projector 13, the infrared exothermic device 14, an operating time adjusting switch 16C for adjusting operation time of the laser generator 17, a projector temperature adjusting switch 16D for adjusting a temperature range of the infrared ray projector 13, an exothermic device temperature adjusting switch 16E for adjusting a temperature range of the infrared exothermic device 14, and an operation display window 16F for displaying the operation status and the operation conditions in digital mode.

FIG. 2 is an enlarged view of the laser generator 17.

Laser employed in the hand skin care apparatus according to the preferred embodiments of the present invention is a low-level soft laser for stimulating human body and has advantages for cellular activation, healing injury, relaxation of inflammation, remove of mold and bacilli causing various diseases, the treatment of housewives eczema, pityriasis, and wrinkle.

Absolutely, a specific wavelength, output power, pulse mode, and pulse parameters must be considered when using the low-level laser according to treatment purposes.

In the present invention, since the laser is not diffused and travels straightly, the projection range of the laser beam generated by a laser light source 17A is diffused through a convex lens 17B and uniformly projected hands. Preferably, as the laser light source 17A, a small and lightweight semiconductor laser is used.

Moreover, the cover 20 is opened and closed to clean the hand accommodating part 30 by removing foreign matter and dust and to keep the hand accommodating part 30 sanitary and clear. The inner wall of the front side 15 is provided with a ceramic wall 22 having a plurality of grooves 23 formed in the longitudinal direction, and the cover 20 is provided with a transparent window 24 at the upper side of the ceramic wall 22 in a predetermined size for a user to directly observe his/her hands in the hand accommodating part 30. The ceramic wall 22 is manufactured by coating ceramic on the composite ceramic body 13C6 or a plastic body to form a ceramic layer.

The ceramic wall 22 emits the far infrared rays and controls irregular emission of air, infrared rays, and laser through the grooves 23 to concentrate the same to hands. Thus, the effects of the hand skin care apparatus are enhanced when using the same.

The hand accommodating part 30 must have a sufficient space to accommodate user's hands and for the user to move his/her hands in the hand accommodating part 30.

The main body 10 and the cover 20 are formed with arc-shaped recesses 41 and 41′, opposite to each other, to form a hand insertion opening 40 at a connection portion between the main body 10 and the cover 20 for guiding the user to insert his/her hands into the hand accommodating part 30. Two hand insertion openings 40, as shown in FIG. 1, may be symmetrically formed at the sides of the man body 10, or, as shown in FIG. 2, a single hand insertion opening 40 may be formed at the central portion of the main body 10. The hand insertion opening 40 must be ergonomically designed such that the user is comfortably serviced by various functions when putting his/her hands in the hand accommodating part 30.

Moreover, the inner circumference of the hand insertion opening 40 is provided with a pair of detecting sensors 50 and 50′ for detecting insertion and withdrawing of hands to control the hand skin care apparatus. The hand skin care apparatus 1 according to the preferred embodiment of the present invention is operated even when one of the detecting sensors 50 and 50′ detects that hands are inserted into the hand accommodating part 30, and is stopped when both hands are withdrawn from the hand accommodating part 30. This operation serves to prevent the skin care apparatus from being deactivated when the user withdraws one hand in order to manipulate the skin care apparatus, or perform other tasks.

A locking structure 60 for controlling the opening and closing of the cover 20 is applied to the cover 20 and the front side 15. Although conventional locking structures can be applied to the cover 20 and the front side 15, in the hand skin care apparatus 1 according to the preferred embodiment of the present invention, the locking structure 60 includes a locking device 63 elastically supported to an end of the cover 20 by a spring 64 and having a handle 61 protruded toward the outside and an insertion guiding slope 62 formed at the lower side thereof, and a locking groove 65 formed at the front side 15, into and from which the locking device 63 is inserted and withdrawn.

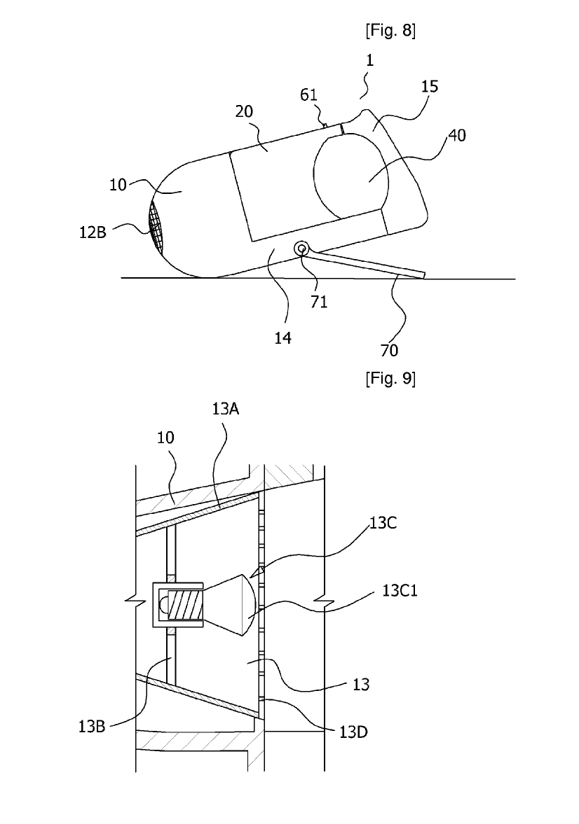

FIGS. 7 and 8 show another preferred embodiments of a titled hand skin care apparatus 1. The hand skin care apparatus in FIG. 7 includes a plate support 70 pivotally hinged to a rear end of the main body 10, and the hand skin care apparatus in FIG. 8 includes a plate support 70 pivotally hinged to an intermediate lower portion of the main body 10.

The plate supports 70 are helpful to slightly lift the hand insertion opening 40 such that the user may easily insert his/her hands into the hand accommodating part 30. Moreover, the user easily manipulate the hand skin care apparatus 1 such that hands are elastically supported when hands are inserted into the hand accommodating part 30.

A torsion spring 72 is installed to a hinge shaft 71 such that the hand skin care apparatus 1 is automatically tilted by the torsion spring 72 over the plate support 70 in normal state, and the torsion spring 72 is deformed when hands are inserted into the hand accommodating part 30 such that the hand skin care apparatus 1 is properly lowered down to the level for the user to keep his/her hands properly and comfortably.

The hand skin care apparatus according to the preferred embodiments of the present invention has no limit for the installation place and method, and for use. The hand skin care apparatus 1 may be fixed or not on a table to use, or may be attached on a wall by a proper fixing device.

Hereinafter, operation of the hand skin care apparatus 1 according to the preferred embodiments of the present invention will be described.

After closing the cover 20, the power switch 16A of the control panel 16 is switched to the “POWER ON” position. At that time, operating time of the suction fan 12A, the infrared ray projector 13, the infrared exothermic device 14, the laser generator 17, and the temperature range of the infrared ray projector 13 and the infrared exothermic device 14 are properly set by the blowing time adjusting switch 16B, the operation time adjusting switch 16C, the projector temperature adjusting switch 16D, and the infrared exothermic device temperature adjusting switch 16E. A user inserts his/her hands through the hand insertion opening 40 and rubs, scrubs, and strokes hands. When user's hands are inserted into the main body 10, the detecting sensors 50 and 50′ at the circumference of the hand insertion opening 40 detect the hand insertion to drive the suction fan 12A. Thus, ambient air is introduced into the main body 10 through the grill 12B. The introduced air is also introduced into and heated by the infrared ray projector 13 heated by applying electric power. The hot air is blown to the hand accommodating part 30 and the infrared ray projector 13 emits infrared rays and far infrared rays toward the hand accommodating part 30 to project the infrared rays and the far infrared rays to the inserted hands. Thus, the hot air dries the inserted hands and the functions and effects of the infrared rays and the far infrared rays are provided to the inserted hands.

At that time, the insulator 13A of the infrared ray projector 13 enhances the effects of the hand skin care apparatus 1 by controlling the heat emission in multiple directions from the infrared ray generator 13C to prevent the main body 10 from being deformed due to overheating and by gathering heat, the infrared rays and the far infrared rays to be concentrated to the hands. The hot air flows along a regular passage of the grooves 23 of the ceramic wall 22 to the hand accommodating part 30 and is concentrated to the hands.

The infrared exothermic device 14 disposed on the bottom of the main body 10 emits heat and the far infrared rays toward the hands when electric power is supplied to the hand skin care apparatus 1, and heats air in the hand accommodating part 30 and the surface member 14D to promote far infrared ray emission. Moreover, the laser generator 1 emits the low-level laser beam toward the hands when the electric power is supplied, thus the functions and effect of the laser beam as described above are provided to the hands.

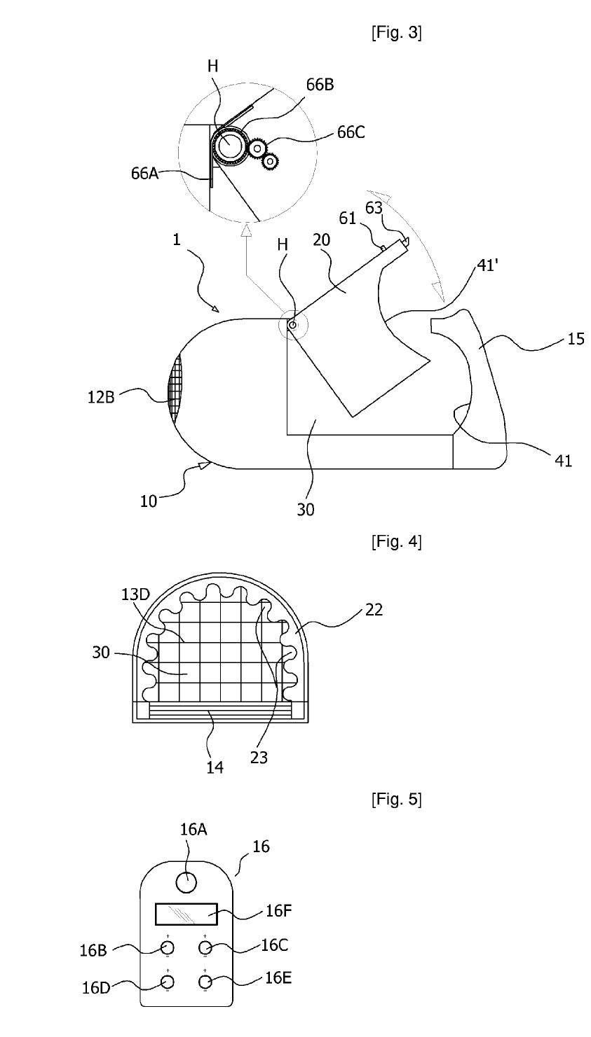

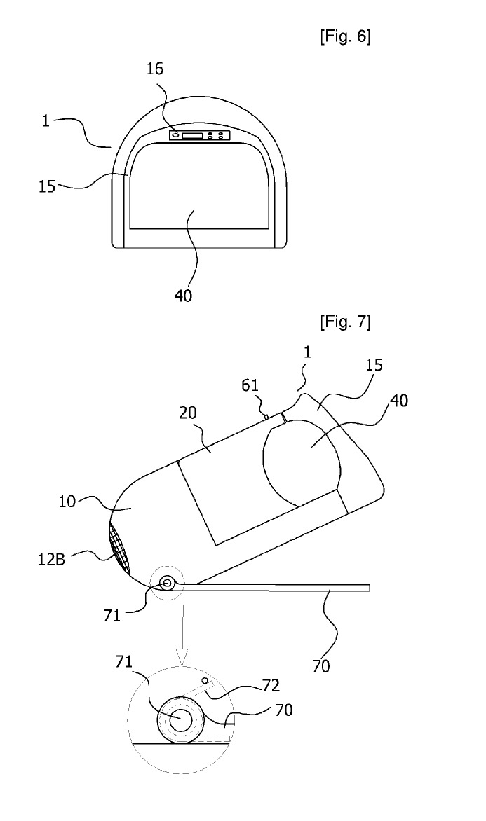

When the hand accommodating part 30 is contaminated due to the introduction of dust, and other foreign matter, or the laser generator 17 is repaired and/or replaced, the locking structure 60 is released to open the cover 20 as shown in FIG. 3.

When pushing the handle 61 of the locking structure 60 provided at the rear side of the cover 20 backward, the locking device 63 compresses the spring 64 and retreats such that a leading end of the locking device 63 is withdrawn from the locking groove 65 of the front side 15, and at this state, the cover 20 is pivoted about the hinge shaft H and easily opened when the cover 20 is lifted.

Referring to FIG. 3, a hinge shaft gear 66B and a torsion spring 66A are installed to the hinge shaft H, wherein the hinge shaft gear 66B is engaged with a reduction gear 66C fixed to the cover 20 or the rear side of the main body 10. When opening the cover 20 by releasing the locking structure 60, the cover 20 is pivoted about the hinge shaft H by the torsion spring 66A and easily lifted, and is slowly and smoothly opened without rapid movement due to the engagement between the hinge shaft gear 66B and the reduction gear 66C.

On the contrary, when the cover 20 is simply pushed down to close the cover 20, the insertion guiding slope 62 of the locking device 63 contacts the circumference of the front side 15 and slides backward and advances due to the elastic force of the spring 64 at the state of alignment of the locking device 63 and the locking groove 65 when the cover is completely closed to be inserted into the locking groove 65, thus locking is completed.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A hand skin care apparatus comprising:
a suction part (12) for sucking ambient air into a main body (10) by a suction fan (12A) and a grill (12B) fixed to a rear inner side of the main body (10);
an infrared ray projector (13) including an infrared ray generator (13C) fixed to an inner central portion of a pipe-shaped insulator (13A) by the pipe-shaped insulator (13A) and a support (13B) to emit heat and infrared rays, and a protective net (13D) installed to the front side of the infrared ray generator (13C);
an infrared exothermic device (14) installed directly below a cover (20) to be connected to the infrared ray projector (13) to emit heat and far infrared rays;
a control panel (16) exposed over the surface of a front side (15) of the main body (10) connected to the infrared exothermic device (14) to control operation of the hand skin care apparatus;
a laser generator (17) fixed in the front side (15) and projecting laser beams on hands;
the cover (20) pivotally coupled to the main body (10) by a hinge shaft (H) to be opened and closed;
a hand accommodating part (30) formed in the cover (20);
a hand insertion opening (40) for guiding a user to insert his/her hands into the hand accommodating part (30);
a pair of detecting sensors (50 and 50′) fixed to the inner circumference of the hand insertion opening (40) to detect the insertion of the hands to control the hand skin care apparatus; and
a locking structure (60) installed to the front side (15) and the cover (20) to selectively lock the cover (20);
wherein the infrared rays, the far infrared rays, and the laser beam are projected to the user's hands inserted into the hand accommodating part (30).

2. The hand skin care apparatus as set forth in claim 1, wherein the infrared ray generator (13C) comprises an infrared ray lamp (13C1) for emitting heat and far infrared rays.

3. The hand skin care apparatus as set forth in claim 1, wherein the infrared ray generator (13C) comprises a halogen lamp (13C3) for emitting heat and far infrared rays.

4. The hand skin care apparatus as set forth in claims 1, wherein the infrared ray generator (13C) is made by winding a heating wire (13C8) around an outer circumference of a quartz glass tube (13C4) accommodating kaoline, yellow soil, or the combination of the kaoline and the yellow soil.

5. The hand skin care apparatus as set forth in claim 1, wherein the infrared ray generator (13C) is made by winding a heating wire (13Cl0) around an outer circumference of a composite ceramic body (13C9) manufactured in the form of a cylinder, a rectangular rod, or a hollow cylinder.

6. The hand skin care apparatus as set forth in claim 1, wherein the main body (10) is formed with a plurality of insertion holes (13C2) formed in regular intervals to insert and install infrared lamps (13C1).

7. The hand skin care apparatus as set forth in claim 3, wherein the halogen lamp (13C3) is disposed in a pipe-shaped composite ceramic body (13C6).

8. The hand skin care apparatus as set forth in claim 1, wherein the infrared exothermic device (14) comprises:
upper and lower insulating plates (14B and 14C);
a heating plate (14A) disposed between the upper and lower insulating plates (14B and 14C);
a surface member (14D) disposed on the upper insulating plate (14B) to emit infrared rays; and
an adiabatic plate (14E) disposed on the lower insulating plate (14C) to block heat transfer.

9. The hand skin care apparatus as set forth in claim 1, wherein the infrared exothermic device (14) comprises:
upper and lower insulating plates (14B and 14C);
a plane heater (14F) disposed between the upper and lower insulating plates (14B and 14C);
a surface member (14D) disposed on the upper insulating plate (14B) to emit infrared rays;
and
an adiabatic plate (14E) disposed on the lower insulating plate (14C) to block heat transfer.

10. The hand skin care apparatus as set forth in claim 1, wherein the infrared exothermic device (14) comprises:
a halogen lamp (14G) attached to the lower surface of a surface member (14D);
and
a reflecting plate (14H) disposed under the halogen lamp (14G); and
an adiabatic member (14I) disposed under the reflecting plate (14H).

11. The hand skin care apparatus as set forth in claim 1, wherein the cover (20) includes a ceramic wall (22) disposed at the inner wall of the cover (20) and having a plurality of grooves (23) formed in the longitudinal direction.

12. The hand skin care apparatus as set forth in claim 1, wherein the cover (20) comprises a transparent window (24) formed at the upper side of the cover (20) in a predetermined size to observe the hands inserted into the hand accommodating part (30).

13. The hand skin care apparatus as set forth in claim 1, wherein the locking structure (60) of the cover (20) comprises:
a locking device (63) including a handle (61) protruded outside and an insertion guiding slope (62);
a spring (64) for returning the locking device (63) to its original position; and
a locking groove (65) formed at the front side (15) and into which the locking device (63) is selectively inserted.

14. The hand skin care apparatus as set forth in claim 1, wherein the hand insertion opening (40) has a pair of hand insertion openings (40) symmetrically formed at front right and left sides of the hand accommodating part (30).

15. The hand skin care apparatus as set forth in claim 1, wherein the hand insertion opening (40) has a single hand insertion opening (40) formed at a central portion of the front side (15) to be communicated to the inside of the hand accommodating part (30).

16. The hand skin care apparatus as set forth in claim 1, further comprising a support (70) coupled to the lower side of the main body (10) by a hinge shaft (71) and a torsion spring (72) to be elastically pivoted.

17. The hand skin care apparatus as set forth in claim 1, wherein the hinge shaft (H) of the main body (10) comprises a hinge shaft gear (66B) and a torsion spring (66A) installed thereto, and the hinge shaft gear (66B) is engaged with a reduction gear (66C) fixed to the cover (20) or the rear side of the main body (10).

18. A hand skin care apparatus comprising:
a suction part (12) for sucking ambient air into a main body (10) by a suction fan (12A) and a grill (12B) fixed to a rear inner side of the main body (10);
an infrared ray projector (13) including an infrared ray generator (13C) fixed to an inner central portion of a pipe-shaped insulator (13A) by the pipe-shaped insulator (13A) and a support (13B) to emit heat and infrared rays, and a protective net (13D) installed to the front side of the infrared ray generator (13C);
an infrared exothermic device (14) installed on the bottom of the main body (10), thereby connected to the infrared ray projector (13) to emit heat and far infrared rays;
a control panel (16) exposed over the surface of a front side (15) of the main body (10) connected to the infrared exothermic device (14) to control operation of the hand skin care apparatus;
a hand accommodating part (30) formed in the main body (10);
a hand insertion opening (40) for guiding a user to insert his/her hands into the hand accommodating part (30); and
a pair of detecting sensors (50 and 50′) fixed to the inner circumference of the hand insertion opening (40) to detect the insertion of the hands to control the hand skin care apparatus;
wherein the infrared rays, the far infrared rays, and the laser beam are projected to the user's hands inserted into the hand accommodating part (30).

19. The hand skin care apparatus as set forth in claim 2, wherein the main body (10) is formed with a plurality of insertion holes (13C2) formed in regular intervals to insert and install infrared lamps (13C1).
